(19) United States
(12) Patent Application Publication
(10) Pub. No.: US 2020/0171311 A1
(43) Pub. Date: Jun. 4, 2020

(54) HYBRID NEUROSTIMULATION LEAD COMBINING FLEXIBLE CIRCUIT ELECTRODES WITH LEAD BODY HAVING BULK CONDUCTOR

(71) Applicant: Verily Life Sciences LLC, South San Francisco, CA (US)

(72) Inventors: Kedar Shah, San Francisco, CA (US); Shiv Sabesan, San Mateo, CA (US); Brian Pepin, Sam Francisco, CA (US)

(73) Assignee: Verily Life Sciences LLC, South San Francisco, CA (US)

(21) Appl. No.: 16/636,401

(22) PCT Filed: Aug. 3, 2018
(86) PCT No.: PCT/US2018/045263

§ 371 Date: Feb. 4, 2020

Related U.S. Application Data
(60) Provisional application No. 62/545,723, filed on Aug. 15, 2017.

(51) Int. Cl. A61N 1/36; A61N 1/375; A61N 1/05

(52) U.S. Cl. CPC A61N 1/36125; A61N 1/0529; A61N 1/3752

(57) ABSTRACT

A device includes a neural interface and a lead body. The lead body includes a bulk conductor and the neural interface includes a flexible circuit. The flexible circuit includes a microfabricated substrate and an exposed electrode. An interconnect region disposed between the electrode and the bulk conductor provides electrical connection between the electrode and the bulk conductor such that electrical signals can be communicated relative to the electrode via the bulk conductor. The interconnect region can be respectively connected to the electrode and bulk conductor by wire-bonding, welding, or other suitable connection methods.

### BACKGROUND

Implantable medical devices can be used for monitoring (e.g., ongoing glucose monitoring) and for stimulation (e.g., to regulate the beating of a heart). Such devices can include electrodes. The electrodes can be placed at a target location for monitoring or stimulation. In a monitoring scenario, the electrodes gather information from the target location and the electronics package processes the information. In a stimulation scenario, the electronics package generates electrical signals that are delivered to the target location via the electrodes.

### SUMMARY

Various examples of the present disclosure are directed to hybrid neural leads (e.g., including (a) a micro-fabricated thin-film electrode array suitable for accommodating complex geometry, and (b) high-reliability bulk wiring within a lead body for conveying signals to or from the electrode array), systems including the same, and methods for forming the same.

In one example, a method of fabricating a neural lead assembly is provided. The method includes providing a lead body including a bulk conductor having a first end and a second end, where the first end is configured for connection with an electronic device. The method further includes providing a flexible circuit including an exposed electrode. The method further includes providing an interconnect region. The method further includes establishing an electrical connection between the interconnect region and the exposed electrode. The method further includes joining the second end of the bulk conductor of the lead body in electrical connection with the interconnect region. As a result, the bulk conductor of the lead body is electrically connected with the exposed electrode via the interconnect region so as to establish a path for travel of signals between the exposed electrode and the first end of the bulk conductor of the lead body.

In another example, a system is provided including a lead body, a flexible circuit, and an interconnect region. The lead body includes a bulk conductor having a first end and a second end. The flexible circuit includes an exposed electrode. The interconnect region is disposed between the exposed electrode and the bulk conductor. The exposed electrode is electrically connected to the interconnect region and the bulk conductor is joined in electrical connection with the interconnect region such that the exposed electrode and bulk conductor are in electrical communication via the interconnect region.

In a further example, a system is provided including a leady body, a flexible circuit, an interconnect region, and a multiplexer. The lead body includes a bulk conductor having a first end and a second end. The flexible circuit includes an exposed electrode. The interconnect region is disposed between the exposed electrode and the bulk conductor. The exposed electrode is electrically connected to the interconnect region and the bulk conductor is joined in electrical connection with the interconnect region such that the exposed electrode and bulk conductor are in electrical communication via the interconnect region. The multiplexer is in electrical connection with the exposed electrode and in electrical connection with the bulk conductor. The multiplexer is configured to control characteristics of signals relative to the exposed electrode in response to input received via the bulk conductor.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 illustrates a top view of a neurostimulation system, according to at least one example.

FIG. 2 shows a cross-sectional view of a lead that may be utilized in the neurostimulation system of FIG. 1, according to at least one example.

FIG. 3 shows a portion of a lead that includes a multiplexer or controller that may be utilized in the neurostimulation system of FIG. 1, according to at least one example.

FIG. 4 shows a portion of a lead that includes a flexible circuit that may be utilized in the neurostimulation system of FIG. 1, according to at least one example.

FIG. 5 shows a portion of a lead that includes a strain-relief feature that may be utilized in the neurostimulation system of FIG. 1, according to at least one example.

FIG. 6 is a flowchart illustrating a process of fabricating a neural lead, according to at least one example.

### DETAILED DESCRIPTION

Various examples described herein are directed to flexible circuits including neural interfaces and combined with associated lead bodies having bulk wiring into a hybrid neural lead in the context of neurostimulation devices and/or monitoring devices. Those of ordinary skill in the art will realize that the following description is illustrative only and is not intended to be in any way limiting. For example, the flexible circuits and/or lead bodies described herein can also be used for other applications in which connections are made between electronic devices and electrodes. In some examples, the flexible circuits and/or lead bodies can be used in applications that are not implanted in human tissue.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

In an illustrative example, a neurostimulation system is implanted into a human body, such as to facilitate monitoring of target tissue and/or to facilitate imparting stimulation to target tissue. The neurostimulation system includes a neural interface, an electronic device, a lead body, and an interconnect part.

The neural interface in this illustrative example is microfabricated from a flexible circuit and includes exposed electrodes that can be placed at different target locations in the human body. The microfabrication of the flexible circuit allows creation of a suitable complex geometry for the electrodes of the flexible circuit to engage or otherwise interface with a nerve or other form of target tissue.

The electronic device (e.g., a neurostimulation device) in this illustrative example is connected with the microfabricated flexible circuit of the neural interface via a suitable conduit for conveying signals between the neural interface and the electronic device (e.g., so that signals from the neural interface can be recorded at the electronic device for monitoring the tissue and/or so that signals can be imparted from the electronic device through the neural interface to stimulate the tissue). The electronic device is significantly larger than the flexible circuit of the neural interface, so the conduit there between is routed through muscle or other tissue between the location at which the neural interface is anchored and a space large enough for the electronic device.

In particular, the conduit connecting the microfabricated flexible circuit of the neural interface with the electronic device in this illustrative example includes the lead body and the interconnect part. The lead body includes bulk wires or bulk conductors that are substantially larger than the electrical connections within the flexible circuit that forms the neural interface. For example, whereas features of the flexible circuit may be on the order of less than 1 micron thick, the bulk conductors may be on the order of greater than 10 microns in thickness. The greater size of the bulk conductors allows the lead body to be more robust than the flexible circuit and exhibit greater fatigue resistance suitable for withstanding forces that may be exerted by movement of the muscle or other tissue through which the lead body is routed between the electronic device and the neural interface.

The interconnect part in this illustrative example provides a transition between the microfabricated flexible circuit of the neural interface and the bulk conductors of the lead body. The interconnect part includes bond pads formed of gold, platinum, or other suitable material. The bond pads are grouped in different sets. The sets of bond pads respectively are positioned at opposite ends of the interconnect part and are connected to one another by metal traces or other electrically conductive paths. One set of the bond pads are wire bonded, welded, or otherwise joined in electrical connection with the bulk conductors of the lead body (e.g., with each bond pad connected to a respective bulk conductor). The other set of bond pads are wire bonded, welded, or otherwise joined in electrical connection with respective contacts for electrodes of the microfabricated flexible circuit of the neural interface. The connection between the sets of bond pads thus electrically connect the bulk conductors of the lead body with the electrodes of the microfabricated flexible circuit of the neural interface and permit signals to travel between the electrodes and the electronic device via the lead body and interconnect part.

The interconnect part in this illustrative example can also include a multiplexer or controller. The multiplexer is situated between the sets of bond pads and can be a circuit, computing device, or any other component that can control routing and/or other characteristics of signals passing between the bulk conductors of the lead body and the electrodes of the microfabricated flexible circuit of the neural interface. For example, in response to power and data signals received from the electronic device through the bulk conductors, the multiplexer can select which among various electrodes will be permitted to provide a monitoring signal back through the bulk conductors to the electronic device or in which sequential order; select which among various electrodes will be permitted to provide a stimulating signal to tissue or in which sequential order; set a positive polarity or negative polarity or set some other variable characteristic of a stimulating signal to be provided by a particular electrode; or implement combinations of these or other functions. Including the multiplexer can accordingly permit the neurostimulation system to function with many controllable channels (e.g., four, eight, tens, hundreds, or more) for electrodes (e.g., which may be microfabricated or otherwise relatively much smaller and easier to include in a concentrated space in comparison to bulk conductors), while at the same time only including a small number of the bulk conductors (e.g., one for power transmission and a second for data transmission). Such use of a multiplexer to allow many electrodes with corresponding few bulk conductors may avoid an arrangement in which bulk conductors are mapped one-to-one with electrodes and may avoid a corresponding result in which the lead body would occupy too much space (accommodating the corresponding large number of bulk conductors) to be viable for implantation in the body.

Reference will now be made in detail to implementations of examples as illustrated in the accompanying drawings. Like reference indicators will be used throughout the drawings and the following description to refer to the same or like items. In many instances, similar elements may be identified by the same reference numeral and differentiated by a different letter suffix in the drawings. Thus in the following text description, elements may be referenced with suffixes (e.g., for referencing individual or specific elements such as a first electrode 212A or a second electrode 212B) or without suffixes (e.g., for generally or collectively referencing elements such as one or more of the electrodes 212).

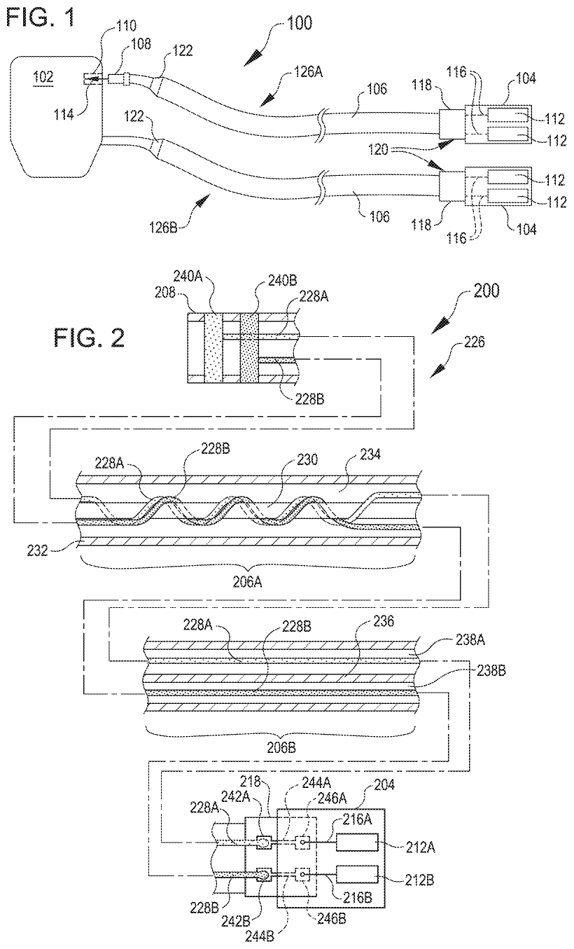

FIG. 1 illustrates a neurostimulation system 100, according to at least one example. The neurostimulation system 100 includes an electronic device 102, a neural interface 104, a lead body 106, and an interconnect part 118 connecting the lead body 106 and the neural interface 104. The lead body 106, the interconnect part 118, and the neural interface 104 can each be respective components of a lead 126. In some examples, respective components of the neurostimulation system 100 (e.g., the electronic device 102 and the lead 126) are implanted in a person's body through one or more incisions.

The depicted neurostimulation system 100 also includes a connector assembly 108, which is configured to mate the detachable lead 126A with the electronic device 102. The depicted connector assembly 108 forms an end of the lead body 106 and functions to provide an interface for establishing connection between the lead body 106 and the electronic device 102. For example, the connector assembly 108 shown in FIG. 1 can be inserted into a socket 110 in the electronic device 102 (e.g., as illustrated by arrow 114) to releasably connect the connector assembly 108 with the electronic device 102. The connector assembly 108 can have any suitable form factor for releasably securing the lead body 106 in electrical connection with the electronic device 102. Examples include, but are not limited to, a planar array of contacts, a cylindrical array of contacts, or other similar contact distribution.

In some examples, a lead body 106 may connect to the electronic device 102 without the connector assembly 108. For example, in FIG. 1, a fixed lead 126B is depicted with a lead body 106 that is hard-wired or otherwise permanently connected with the electronic device 102 rather than releasably secured by a connector assembly 108. The fixed lead 126B may be utilized in addition to or in lieu of the detachable lead 126A associated with the connector assembly 108. The lead body 106, neural interface 104, and other components of the depicted fixed lead 126B are substantially the same as those for the detachable lead 126A associated with the connector assembly 108. Accordingly, it is understood that reference herein to respective elements of a lead 126 may correspond to either the detachable lead 126A or to the fixed lead 126B.

The electronic device 102 can be any suitable active implantable device such as those for neuromodulation or neurostimulation. Examples of such devices include deep brain stimulators, cochlear implants, cardiac pacemakers, bioelectric devices, peripheral nerve stimulation systems, and other similar devices. In some examples, the electronic device 102 is a monitoring device. For example, the electronic device 102 can be attached to the neural interface 104 (e.g., via the lead body 106) in order to monitor conditions of a patient's health. Examples of such devices include those used for glucose monitoring. Such devices may also include those used for glucose monitoring and delivery.

The neural interface 104 is formed as a flexible circuit (e.g., flex circuit, flexible printed circuit board, flex print, or other similar flexible circuit). In some examples, the neural interface 104 is formed using a microfabrication technique. Thus, the neural interface 104 can be a flexible microfabricated circuit board. The neural interface 104 can be formed from polyimide, paraben, liquid crystal polymer, polyether ether ketone (PEEK), plain polyester film (PEP), or any other similar material.

The neural interface 104 includes an array of electrodes 112. Each of the electrodes 112 can be placed at one or many target locations within the patient's nerves, depending on the implementation. While the array of electrodes 112 is shown as an electrode cuff, it is understood that the electrodes 112 may take other form factors, including, for example, separate electrodes that can be spaced and placed separate from each other. The dimensions of the electrodes 112 can vary depending on the application. The neural interface 104 may also be in the geometry of a cuff around the nerves, such as a longitudinal intrafascicular interface or a transverse intrafascicular interface.

A coating 120 is shown in FIG. 1 at least partially covering the lead 126. The coating 120 can be applied using one or more of a variety of processes (e.g., dip coating, cast molding, heat shrinking, and other similar processes). The coating 120 may provide additional mechanical bulk for handling and/or for abrasion resistance in vivo. The coating 120 may be formed from flexible material such as silicone polymers (e.g., medical grade silicones) and other similarly flexible materials that also have biocompatibility. Although the coating 120 in FIG. 1 is depicted as uniformly applied along the lead 126, different portions of the lead 126 may differ from one another in thickness, material, or other characteristics of the coating 120. In FIG. 1, respective portions of the lead body 106 and of the interconnect part 118 are partially obscured by the presence of the coating 120. Accordingly, the interconnect part 118 and lead body 106 will be generally described with respect to FIG. 1, while examples of other feature that may be included or implemented in association with the interconnect part 118 and/or lead body 106 will be described below with reference to other figures in which the coating 120 does not obscure view.

The interconnect part 118 provides a transition and electrical connection between the lead body 106 and the neural interface 104. For example, the interconnect part 118 can include suitable structure for connecting electrical conduits of different sizes and/or types, such as connecting microfabricated components of the neural interface 104 with bulk materials of the lead body 106. In FIG. 1, conductive traces 116 from the respective electrodes 112 of the neural interface 104 are shown extending toward the interconnect part 118. The interconnect part 118 can electrically connect the conductive traces 116 with the lead body 106 to permit travel of signals along the lead 126 between the electrodes 112 and the electronic device 102. The interconnect part 118 can be a region of the neural interface 104 (e.g., a region of a flexible circuit), or the interconnect part 118 may be a part that is distinct form—but joined with—the neural interface 104.

The depicted neurostimulation system 100 also includes a strain-relief feature 122. In some examples, the strain-relief feature 122 may correspond to a tapered structure, e.g., formed by molding a tapered elastomeric material from a relatively more stiff region of one component to a relatively more flexible region of another component. In this manner, the tapered elastomeric material may provide strain relief at areas where stiff regions connect to flexible regions. Such a transition zone may prevent concentration of stresses at the transition point between the two regions that might lead to mechanical failure For example, the strain-relief feature 122 depicted in FIG. 1 corresponds to a taper from the relatively more stiff connector assembly 108 to the relatively more flexible lead body 106. Other methods and associated structures for strain relief may also be utilized, such as that described below with reference to FIG. 5. Moreover, structures for strain relief are not limited to placement near where a lead body 106 connects to an electronic device, but may additionally or alternatively be placed near where a lead body connects to an interconnect part 118 or at various other locations along the lead 126.

FIG. 2 illustrates a cutaway view of parts of a neurostimulation system 200, according to at least one example. The neurostimulation system 200 is an example of the neurostimulation system 100 of FIG. 1. In the illustrated embodiment, the neurostimulation system 200 includes a lead 226 that includes features that are similar to features of like names and reference numbers from FIG. 1, and, as such, description of various aspects of these features are not repeated.

The lead body 206 shown in FIG. 2 includes bulk conductors 228. Specifically, a first bulk conductor 228A and a second bulk conductor 228B are shown in FIG. 2, although the lead body 206 may include any number of bulk conductors 228, including one, two, or more than two. The bulk conductors 228 can take any suitable form factor and can be formed from any suitable bio compatible conductive material such as gold, titanium, platinum, iridium, niobium, platinum alloy, iridium alloy, nickel titanium alloy, nickel-cobalt-chromium-molybdenum alloy (e.g., MP35N®), or some other similar or otherwise suitable material. The bulk conductors 228 can be coated with a flexible, insulative material such as silicon, polyurethane, Teflon®, or some other similar or otherwise suitable material. The bulk conductors 228 can be microwires. In some examples, the bulk conductors 228 may be wires or other structures that are fabricated by drawing through a hole in a die or draw plate or fabricated from other processes that may differ from the microfabrication process that can be utilized to form the flexible circuit of the neural interface 204. In some examples, the bulk conductors 228 are distinguishable on the basis of size from features of the neural interface 204. For example, the bulk conductors 228 may be greater than ten microns in thickness in contrast to conductive traces 216 of the neural interface 204 that may be less than one micron thick. In some examples, the bulk conductors 228 have a size (e.g., thickness) that is at least ten times that of a comparable size of a conductive trace 216 or other relevant feature of the neural interface 204.

The bulk conductors 228 can be arranged in any suitable manner along the length of the lead body 206. As one example, in a first lead body portion 206A shown in FIG. 2, the bulk conductors 228 are wrapped around a mandrel 230 (e.g., which may be non-conductive) and occupy space in an annulus 234 between the mandrel 230 and a casing 232 of the lead body 206. The bulk conductors 228 can be electrically isolated from one another (e.g., to allow distinct signals to be carried through the different bulk conductors 228), such as by the inclusion of insulative coatings on one or more of the bulk conductors 228A or 228B or by spacing the coils of the different bulk conductors 228A and 228B apart from one another by a sufficient amount along the mandrel 230 to prevent electrical conductive interaction between the separate bulk conductors 228A and 228B. As another example, in a second lead body portion 206B shown in FIG. 2, the bulk conductors 228 are routed respectively through separate lumens 238A and 238B formed by internal walls 236 positioned within the second lead body portion 206B. In some examples, the separate lumens 238 can have insulated boundaries that can prevent electrical contact between bulk conductors 228 that do not have respective insulative coatings. Although FIG. 2 illustrates an arrangement with both wrapping about a mandrel 230 and routing through lumens 238 in the same lead body 206, these or other routing arrangements of bulk conductors 228 may be utilized individually or in other combinations or arrangements.

In FIG. 2, the different bulk conductors 228A and 228B are electrically coupled (e.g., at a first or proximal end) to respective separate contacts 240A and 240B in the connector assembly 208. The contacts 240 can mate with respective features in the electronic device 102 to facilitate transfer of signals to or from the electronic device 102 via the lead body 206. The contacts 240 may correspond to planar, cylindrical, or other shapes for releasable engagement or for permanent engagement.

The bulk conductors 228 in FIG. 2 are also shown coupled (e.g., at a second or distal end) to an interconnect part 218. The interconnect part 218 in FIG. 2 includes a substrate that is distinct from the flexible circuit that forms the neural interface 204. In particular, in the arrangement in FIG. 2, the flexible circuit that forms the neural interface 204 is shown overlaying a portion of the interconnect part 218. The substrate of the interconnect part 218 can include ceramic material or other forms of non-conductive or suitable material.

The interconnect part 218 in FIG. 2 includes a first set of bond pads 242 and a second set of bond pads 246 connected by respective electrically conductive paths 244 (e.g., formed by metal traces or other conductive material on the interconnect part 218). The first set of bond pads 242 can facilitate connection of the bulk conductors 228 with the interconnect part 218, the second set of bond pads 246 can facilitate connection of electrodes 212 of the neural interface 204 with the interconnect part 218, and the conductive paths 244 between the first set of bond pads 242 and the second set of bond pads 246 can facilitate travel of signals between the respectively connected bulk conductors 228 and the neural interface 204.

The bulk conductors 228 can be connected to the first set of bond pads 242 by any suitable method, and the neural interface 204 can be connected to the second set of bond pads 246 by any suitable method. Examples may include resistance welding, conductive epoxy, thermosonic welding, mechanical crimping, laser welding, and/or any other suitable operation. Moreover, the exposed electrodes 212, the conductive traces 216 the first set of bond pads 242, the electrically conductive path 244, and the second set of bond pads 246 can be formed from any suitable bio-compatible conductive material such as gold, titanium, platinum, iridium, niobium, platinum alloy, iridium alloy, nickel titanium alloy, nickel-cobalt-chromium-molybdenum alloy (e.g., MP35N®), or any other suitable material. However, to aid understanding, some more specific illustrative examples will now be described.

In a first illustrative example, the interconnect part 218 is a ceramic substrate on which the first set of bond pads 242 are formed from platinum material. The bulk conductors 228 correspond to microwires formed of platinum or a platinum iridium blend and having a size ranging between 75 and 125 microns. Each bulk conductor 228 is resistance welded, laser welded, or otherwise welded to a respective bond pad in the first set of bond pads 242.

In a second illustrative example, the interconnect part 218 is a ceramic substrate on which the first set of bond pads 242 are formed from platinum material.

The bulk conductors 228 correspond to microwires formed of gold and having a size ranging between 75 and 125 microns. Each bulk conductor 228 is wire-bonded (e.g., via controlled application of heat, pressure, and ultrasonic energy) to a respective bond pad in the first set of bond pads 242.

In a third illustrative example, the interconnect part 218 is a ceramic substrate on which the second set of bond pads 246 are formed from gold material. The neural interface 204 includes conductive traces 216 of gold that range in size from 0.1 to 0.9 microns. Respective conductive traces 216 are wire bonded to a respective bond pad in the second set of bond pads 246.

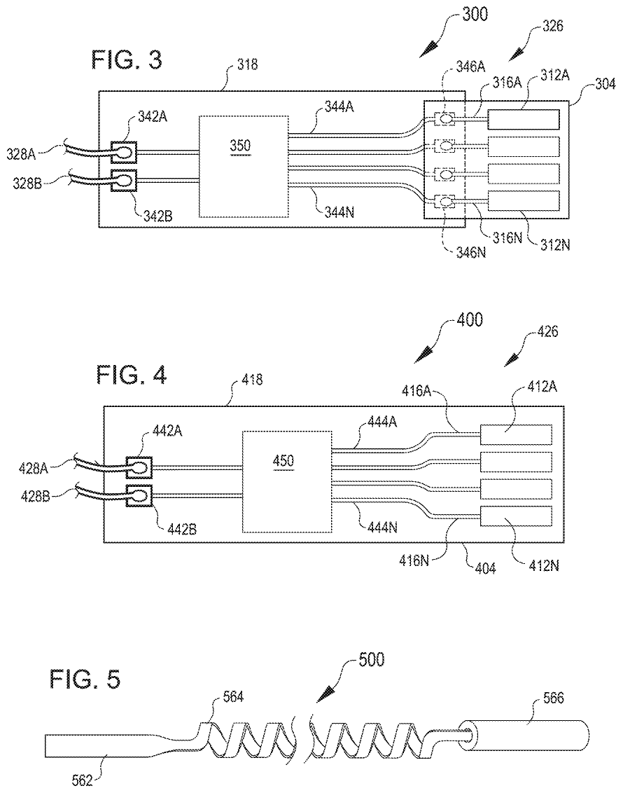

FIG. 3 illustrates a cutaway view of parts of a neurostimulation system 300, according to at least one example. The neurostimulation system 300 is an example of the neurostimulation system 100 of FIG. 1. In the illustrated embodiment, the neurostimulation system 300 includes a lead 326 that includes features that are similar to features of like names and reference numbers from FIG. 1 and/or FIG. 2, and, as such, description of various aspects of these features are not repeated.

The lead 326 in FIG. 3 includes a controller or a multiplexer 350. The multiplexer 350 shown in FIG. 3 is positioned on the interconnect part 318. In FIG. 3, the interconnect part 318 includes a substrate that is distinct from the flexible circuit that forms the neural interface 304.

The multiplexer 350 can include a circuit or circuitry to provide associated functions. In some examples, the multiplexer 350 can include a computing device that receives input from one or more elements of the system 300 and provides output to the same or other elements of the system 300. For example, the computing device can include a processor and memory. The processor may be implemented as appropriate in hardware, computer-executable instructions, firmware, or combinations thereof. The memory may include any suitable form of non-transitory computer-readable medium. The memory can include instructions which are generally executed by the processor for implementing the features disclosed herein. Computer-executable instruction or firmware implementations of the processor may include computer-executable or machine-executable instructions written in any suitable programming language to perform the various functions described. The memory in various examples can store information from input provided to the computing device from other elements of the system 300, which may allow the information to be later accessed and/or further processed.

The multiplexer 350 can control routing and/or other characteristics of signals passing between the bulk conductors 328 and the electrodes 312 of the neural interface 304. To this end, the multiplexer 350 in FIG. 3 is communicatively coupled with a first bulk conductor 328A and a second bulk conductor 328B (e.g., via respective bond pads 342A and 342B), although a different number of bulk conductors 328 could be used, including one, two, three, or more than three. The multiplexer 350 in FIG. 3 is also communicatively coupled with a plurality of N electrodes 312A-312N (e.g., via respective bond pads 346A-346N). Although the number N of electrodes 312 is four in FIG. 3, any number N of electrodes 312 could be utilized.

The multiplexer 350 can utilize any of the bulk conductors 328 and any of the electrodes 312 as respective inputs and/or outputs. For example, in FIG. 3, the multiplexer 350 can receive power and data or control signals through the bulk conductors 328. Based on the control signals received through the bulk conductors 328, the multiplexer 350 may control how the power from the bulk conductors 328 is provided to the electrodes 312 (e.g., in a stimulating mode). Additionally or alternatively, based on the control signals received through the bulk conductors 328, the multiplexer 350 may control how signals are provided from the electrodes 312 (e.g., in a monitoring mode) to the bulk conductors 328. Various functions of the multiplexer 350 accordingly may be implemented individually or in combination.

In some examples, the multiplexer 350 selects which among the various electrodes 412 will be permitted to provide a stimulating signal to tissue. In an illustrative example, the first bulk conductor 328A provides power to the multiplexer 350, and the second bulk conductor 328B provides a control input. Based on the control input from the second bulk conductor 328B, the multiplexer 350 regulates at least a portion of the power input from the first bulk conductor 328A toward only the first electrode 412A without routing power toward the final electrode 412N.

In some examples, the multiplexer 350 determines a sequential order in which electrodes 412 will be permitted to provide a stimulating signal to tissue. In an illustrative example, the first bulk conductor 328A provides power to the multiplexer 350, and the second bulk conductor 328B provides a control input. Based on the control input from the second bulk conductor 328B, the multiplexer 350 regulates at least a portion of the power input from the first bulk conductor 328A toward the first electrode 412A for a first interval, toward the final electrode 412N for a second interval, and toward the first electrode 412A again for a third interval, e.g., to provide a particular stimulation pattern to the target tissue.

In some examples, the multiplexer 350 sets a positive polarity or negative polarity or sets some other variable characteristic of a stimulating signal to be provided by a particular electrode 412. In an illustrative example, the first bulk conductor 328A provides power to the multiplexer 350, and the second bulk conductor 328B provides a control input. Based on the control input from the second bulk conductor 328B, the multiplexer 350 regulates at least a portion of the power input from the first bulk conductor 328A to affect the polarity, amplitude, frequency, or other variable characteristic of a stimulating signal communicated to a particular electrode 412. The multiplexer 350 may adjust such a variable characteristic of a stimulating signal according to a specified pattern to provide a particular stimulation pattern to the target tissue.

In some examples, the multiplexer 350 selects which among the various electrodes 412 will be permitted to provide a monitoring signal from the tissue. In an illustrative example, the first bulk conductor 328A provides a control input to the multiplexer 350, and the second bulk conductor 328B provides a monitoring output. Based on the control input from the first bulk conductor 328A, the multiplexer 350 permits a monitoring signal from the first electrode 312A to be conveyed along the second bulk conductor 328B without allowing a monitoring signal from the final electrode 412N to also be conveyed. For example, such an arrangement may correspond to the multiplexer 350 responding to a query from the electronic device 102 (FIG. 1) for a monitoring signal for a particular location (e.g., associated with the first electrode 312A).

In some examples, the multiplexer 350 determines a sequential order in which electrodes 412 will be permitted to provide a monitoring signal from the tissue. In an illustrative example, the first bulk conductor 328A provides a control input to the multiplexer 350, and the second bulk conductor 328B provides a monitoring output. Based on the control input from the first bulk conductor 328A, the multiplexer 350 regulates signal propagation along the second bulk conductor 328B to reflect a monitoring signal from the first electrode 412A for a first interval, reflect a monitoring signal from the final electrode 412N for a second interval, and reflect a monitoring signal from the first electrode 412A again for a third interval, e.g., to provide a particular monitoring pattern from different locations of the target tissue.

The system 300 is not limited to the specific examples described above. For example, although some examples include power being transmitted along one bulk conductor 328 and data being transmitted along a separate bulk conductor 328 relative to the multiplexer 350, in some examples, power and data may be transmitted relative to the multiplexer 350 along a shared or single bulk conductor 328. Moreover, one bulk conductor may 328 be utilized to both send and receive data relative to the multiplexer 350, or the multiplexer 350 may be coupled with a bulk conductor 328 to send data and a separate bulk conductor 328 to receive data.

FIG. 4 illustrates a cutaway view of parts of a neurostimulation system 400, according to at least one example. The neurostimulation system 400 is an example of the neurostimulation system 100 of FIG. 1. In the illustrated embodiment, the neurostimulation system 400 includes a lead 426 that includes features that are similar to features of like names and reference numbers from FIG. 1, FIG. 2, and/or FIG. 3, and, as such, description of various aspects of these features are not repeated.

The lead 426 in FIG. 4 is similar to the lead 326 of FIG. 3. However, unlike in FIG. 3, the interconnect part 418 in FIG. 4 forms a part of (e.g., is not separate from) the flexible circuit that forms the neural interface 404. Although the multiplexer 450 in FIG. 4 is shown on an interconnect part 418 that forms part of the flexible circuit of the neural interface 404 and the multiplexer 350 in FIG. 3 is shown on an interconnect part 318 that is distinct from the flexible circuit of the neural interface 304, a multiplexer may additionally or alternatively be positioned on a flexible circuit that is separate from an interconnect part 418.

FIG. 5 illustrates a strain relief feature 500, according to at least one example. The strain relief feature 500 can include a flex circuit portion 562, a coil 564, and a wire portion 566. The strain relief feature 500 may be included, e.g., in lieu of the strain-relief feature 122 described above with respect to FIG. 1. The coil 564 may be an extension of the flex circuit portion 562 that has been twisted and thermo-formed to hold the shape of the coil 564. The shape of the coil 564 may permit the strain relief feature 500 to be extensible and thus relieve strain that might otherwise be exerted on the flex circuit portion 562 and/or the wire portion 566. In some examples, the respective flex circuit portion 562 and the wire portion 566 may be bonded to other portions of the lead 126 using methods described herein to provide electrical connections between respective elements joined by the strain relief feature.

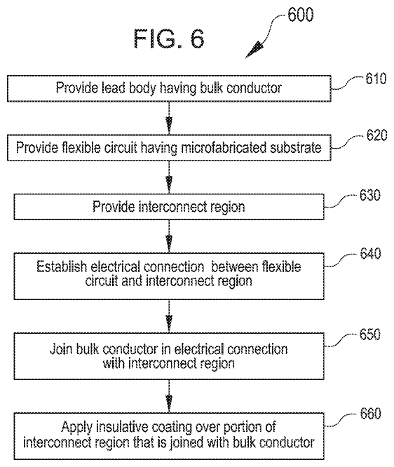

FIG. 6 is a flowchart illustrating a process 600 of fabricating a neural lead (e.g., leads 126, 226, 326, 426, or 526), according to at least one example.

At 610, the process 600 can include providing a lead body that includes a bulk conductor. For example, the bulk conductor may correspond to any of the bulk conductors 228, 328, or 428 as respectively described above with respect to FIGS. 2, 3, and 4. The bulk conductor may be elongate or otherwise extend between a first end and a second end of the bulk conductor, where the first end corresponds to a connector assembly 108 or 208 (e.g., FIG. 1 or FIG. 2) or is otherwise configured for connection with an electronic device such as the electronic device 102 (e.g., FIG. 1).

At 620, the process 600 can include providing a flexible circuit having a microfabricated substrate. The flexible circuit may define a neural interface (e.g., any of the neural interfaces 104, 204, 304, or 404 as respectively described above with respect to FIGS. 1, 2, 3, and 4). For example, the neural interface may include an exposed electrode (e.g., any of the electrodes 112, 212, 312, or 412 as respectively described above with respect to FIGS. 1, 2, 3, and 4).

At 630, the process 600 can include providing an interconnect region. The interconnect region can include metal or other electrically conductive material arranged in an electrically conductive path between a first point (e.g., for connecting to the flexible circuit) and a second point (e.g., for connecting to the bulk conductor). In some examples, providing the interconnect region involves providing an interconnect substrate that includes the interconnect region and is distinct from the flexible circuit (e.g., as described above for the interconnect parts 218 and 318 with respect to FIGS. 2-3). In other examples, providing the interconnect region involves providing the interconnect region as a portion of the microfabricated substrate or other part of the flexible circuit (e.g., as described above for the interconnect part 418 with respect to FIG. 4).

At 640, the process 600 can include establishing electrical connection between the flexible circuit and the interconnect region. This may correspond to establishing electrical connection between the interconnect region and the electrode of the neural interface. Establishing the electrical connection at 640 may include establishing an electrical connection between the first point of the electrically conductive path of the interconnect region and the electrode of the neural interface.

In examples in which the interconnect region corresponds to an interconnect region that is distinct from the flexible circuit (e.g., as described above for the interconnect parts 218 and 318 with respect to FIGS. 2-3), establishing electrical connection between the flexible circuit and the interconnect region may involve joining the flexible circuit in electrical connection with the interconnect region of the interconnect substrate. As an illustrative example with reference to FIG. 2, this may entail joining the flexible circuit of the neural interface 204 in electrical connection with the interconnect region 218 by joining effected via the bond pads 246 through wire-bonding, welding, and/or any other suitable operation described with respect to FIG. 2.

In examples in which the interconnect region corresponds to an interconnect region that is a portion of the microfabricated substrate or other part of the flexible circuit (e.g., as described above for the interconnect part 418 with respect to FIG. 4), establishing electrical connection between the flexible circuit and the interconnect region may involve providing the interconnect region in electrical connection with the electrode, microfabricated substrate, or other part of the flexible circuit. As an illustrative example with reference to FIG. 4, this may entail the flexible circuit of the neural interface 404 including the interconnect part 418 and respective bond pads 442 that are in electrical connection (e.g., via traces 444) with the electrodes 412.

At 650, the process 600 can include joining the bulk conductor of the lead body in electrical connection with the interconnect region. Joining at 650 may include joining the second end of the bulk conductor of the lead body in electrical connection with the second point of the electrically conductive path of the interconnect region. As an illustrative example with reference to FIG. 2, this may entail joining the bulk conductor 228 in electrical connection with the interconnect region 218 by joining effected via the bond pads 242 through wire-bonding, welding, and/or any other suitable operation described with respect to FIG. 2.

As a result of the operations of the process at 610-650, the bulk conductor of the lead body may be electrically connected with the electrode of the neural interface via the interconnect region so as to establish a path for travel of signals between the electrode and the first end of the bulk conductor of the lead body. For example, this may allow signals to travel through the first end of the bulk conductor relative to the electronic device. Examples of the electronic device can include a pulse generator or other component for receiving, transmitting, or receiving and transmitting electrical signals via the bulk conductor relative to the electrode, neural interface, or other portion of the flexible circuit.

At 660, the process can include applying an insulative coating at least over a portion of the interconnect region that is joined with the bulk conductor. As an illustrative example, in FIG. 1, the leads 126 are shown having a coating 120 that obscures features of the leads 126 from sight.

In some aspects, a device, a system, or a method is provided according to one or more of the following examples or according to some combination of the elements thereof. In some aspects, a device or a system described in one or more of these examples can be utilized to perform a method described in one of the other examples or vice versa.

Example # 1. A method of fabricating a neural lead assembly, which may incorporate features of any of the subsequent examples, the method comprising:
providing a lead body comprising a bulk conductor having a first end and a second end, the first end being configured for connection with an electronic device;
providing a flexible circuit comprising an exposed electrode;
providing an interconnect region;
establishing an electrical connection between the interconnect region and the exposed electrode; and
joining the second end of the bulk conductor of the lead body in electrical connection with the interconnect region;
whereby the bulk conductor of the lead body is electrically connected with the exposed electrode via the interconnect region so as to establish a path for travel of signals between the exposed electrode and the first end of the bulk conductor of the lead body.

Example # 2. The method of Example # 1, or any of the preceding or subsequent examples, wherein the flexible circuit defines a neural interface and comprises a microfabricated substrate and the exposed electrode.

Example # 3. The method of Example # 1, or any of the preceding or subsequent examples, wherein the interconnect region comprises metal or other electrically conductive material arranged in an electrically conductive path between a first point and a second point;
wherein the establishing an electrical connection between the interconnect region and the exposed electrode comprises establishing a first electrical connection between the first point of the electrically conductive path of the interconnect region and the exposed electrode; and
wherein the joining the second end of the bulk conductor of the lead body in electrical connection with the interconnect region comprises joining the second end of the bulk conductor of the lead body in a second electrical connection with the second point of the electrically conductive path of the interconnect region.

Example # 4. The method of Example # 3, or any of the preceding or subsequent examples, further comprising
applying a first insulative coating over the first electrical connection that connects the first point of the electrically conductive path of the interconnect region with the exposed electrode; and
applying a second insulative coating over the second electrical connection that connects the second point of the electrically conductive path of the interconnect region with the lead body.

Example # 5. The method of Example # 1, or any of the preceding or subsequent examples, wherein the providing the interconnect region comprises providing an interconnect substrate distinct from the flexible circuit, the interconnect substrate comprising the interconnect region; and
wherein the establishing an electrical connection between the interconnect region and the exposed electrode comprises joining the flexible circuit in electrical connection with the interconnect region of the interconnect substrate.

Example # 6. The method of Example # 1, or any of the preceding or subsequent examples, wherein the providing the interconnect region and the establishing the electrical connection between the interconnect region and the exposed electrode comprises providing the interconnect region as a portion of the flexible circuit and in electrical connection with the exposed electrode.

Example # 7. The method of Example # 1, or any of the preceding or subsequent examples, wherein the electronic device comprises a component for receiving, transmitting, or receiving and transmitting electrical signals via the bulk conductor.

Example # 8. The method of Example # 1, or any of the preceding or subsequent examples, wherein the component comprises a pulse generator.

Example # 9. The method of Example # 1, or any of the preceding or subsequent examples, further comprising:
applying an insulative coating at least over a portion of the interconnect region that is joined with the second end of the bulk conductor.

Example # 10. The method of Example # 1, or any of the preceding or subsequent examples, wherein the bulk conductor has a thickness at least ten times that of a conductive trace of the flexible circuit.

Example # 11. The method of Example # 1, or any of the preceding or subsequent examples, wherein the bulk conductor has a thickness greater than ten microns and the flexible circuit has a conductive trace having a thickness of less than one micron.

Example # 12. The method of Example # 1, or any of the preceding or subsequent examples, wherein the joining the second end of the bulk conductor of the lead body in electrical connection with the interconnect region is accomplished by welding the second end of the bulk conductor to a bond pad of the interconnect region.

Example # 13. The method of Example # 12, or any of the preceding or subsequent examples, wherein the welding comprises laser welding.

Example # 14. The method of Example # 12, or any of the preceding or subsequent examples, wherein the welding comprises resistance welding.

Example # 15. The method of Example # 12, or any of the preceding or subsequent examples, wherein the welding comprises ultrasonic welding.

Example # 16. The method of Example # 1, or any of the preceding or subsequent examples, wherein the joining the second end of the bulk conductor of the lead body in electrical connection with the interconnect region is accomplished by wire-bonding the second end of the bulk conductor to a bond pad of the interconnect region.

Example # 17. The method of Example # 1, or any of the preceding or subsequent examples, wherein the establishing an electrical connection between the interconnect region and the exposed electrode is accomplished by wire-bonding the interconnect region with the flexible circuit.

Example # 18. The method of Example # 1, or any of the preceding or subsequent examples, wherein the interconnect region comprises a multiplexer;
wherein the multiplexer is in electrical connection with the exposed electrode as a result of the establishing the electrical connection between the interconnect region and the exposed electrode;
wherein the multiplexer is in electrical connection with the bulk conductor as a result of the joining the second end of the bulk conductor of the lead body in electrical connection with the interconnect region; and
wherein the multiplexer is configured to control routing or other characteristics of signals relative to the exposed electrode in response to input received via the bulk conductor.

Example # 19. A system, which may incorporate features of any of the preceding or subsequent examples, comprising:
a lead body comprising a bulk conductor having a first end and a second end;
a flexible circuit comprising an exposed electrode; and
an interconnect region disposed between the exposed electrode and the bulk conductor, wherein the exposed electrode is electrically connected to the interconnect region and the bulk conductor is joined in electrical connection with the interconnect region such that the exposed electrode and bulk conductor are in electrical communication via the interconnect region.

Example # 20. The system of Example # 19, or any of the preceding or subsequent examples, further comprising an electronic device coupled with the lead body for at least one of sending or receiving signals relative to the exposed electrode via the bulk conductor of the lead body.

Example # 21. The system of Example # 19, or any of the preceding or subsequent examples, wherein the lead body comprises at least one of:
a plurality of lumens each containing a respective bulk conductor; or
a plurality of bulk conductors coiled about a mandrel.

Example # 22. The system of Example # 19, or any of the preceding or subsequent examples, further comprising an interconnect substrate distinct from the flexible circuit, the interconnect substrate comprising the interconnect region.

Example # 23. The system of Example # 19, or any of the preceding or subsequent examples, wherein the interconnect region comprises a portion of the flexible circuit.

Example # 24. The system of Example # 19, or any of the preceding or subsequent examples, further comprising a strain-relief feature comprising a coiled portion disposed between the exposed electrode and the bulk conductor.

Example # 25. The system of Example # 19, or any of the preceding or subsequent examples, further comprising a multiplexer in electrical connection with the exposed electrode and in electrical connection with the bulk conductor, wherein the multiplexer is configured to control characteristics of signals relative to the exposed electrode in response to input received via the bulk conductor.

Example # 26. A system, which may incorporate features of any of the preceding or subsequent examples, comprising:
a lead body comprising a bulk conductor having a first end and a second end;
a flexible circuit comprising an exposed electrode;
an interconnect region disposed between the exposed electrode and the bulk conductor, wherein the exposed electrode is electrically connected to the interconnect region and the bulk conductor is joined in electrical connection with the interconnect region such that the exposed electrode and bulk conductor are in electrical communication via the interconnect region; and
a multiplexer in electrical connection with the exposed electrode and in electrical connection with the bulk conductor, wherein the multiplexer is configured to control characteristics of signals relative to the exposed electrode in response to input received via the bulk conductor.

Example # 27. The system of Example # 26, or any of the preceding or subsequent examples, wherein the bulk conductor is configured to provide power and control signals to the multiplexer.

Example # 28. The system of Example # 26, or any of the preceding or subsequent examples, wherein the bulk conductor comprises a first bulk conductor configured to provide power to the multiplexer and a second bulk conductor configured to provide control signals to the multiplexer.

Example # 29. The system of Example # 26, or any of the preceding or subsequent examples, wherein the flexible circuit comprises a plurality of electrodes; and
wherein the multiplexer is configured to control routing of stimulation signals among the plurality of electrodes based on input received through the bulk conductor.

Example # 30. The system of Example # 26, or any of the preceding or subsequent examples, wherein the flexible circuit comprises a plurality of electrodes; and
wherein the multiplexer is configured to control routing of monitoring signals from among the plurality of electrodes based on input received through the bulk conductor.

Example # 31. The system of Example # 26, or any of the preceding or subsequent examples, wherein the flexible circuit comprises a plurality of electrodes; and
wherein the multiplexer is configured to control a sequential order of transmission of stimulation signals among the plurality of electrodes based on input received through the bulk conductor.

Example # 32. The system of Example # 26, or any of the preceding or subsequent examples, wherein the flexible circuit comprises a plurality of electrodes; and
wherein the multiplexer is configured to control a sequential order of transmission of monitoring signals from among the plurality of electrodes based on input received through the bulk conductor.

Example # 33. The system of Example # 26, or any of the preceding or subsequent examples, wherein the multiplexer is configured to control a polarity or other variable characteristic of a stimulation signal to the exposed electrode based on input received through the bulk conductor.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure. For example, more or fewer steps of the processes described herein may be performed according to the present disclosure. Moreover, other structures may perform one or more steps of the processes described herein.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Some examples in this disclosure may include a processor. A computer-readable medium, such as RAM may be coupled to the processor. The processor can execute computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices, such as programmable logic controllers (PLCs), programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example, computer-readable storage media, that may store instructions that, when executed by the processor, can cause the processor to perform the steps described herein as carried out, or assisted, by a processor. Examples of computer-readable media may include, but are not limited to a memory chip, ROM, RAM, ASIC, or any other medium from which a computer processor can read or write information. The processor, and the processing described, may be in one or more structures, and may be dispersed through one or more structures. The processor may comprise code for carrying out one or more of the methods (or parts of methods) described herein.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

That which is claimed is:

1. A method of fabricating a neural lead assembly, the method comprising:
providing a lead body comprising a bulk conductor having a first end and a second end, the first end being configured for connection with an electronic device;
providing a flexible circuit comprising an exposed electrode;
providing an interconnect region;
establishing an electrical connection between the interconnect region and the exposed electrode; and
joining the second end of the bulk conductor of the lead body in electrical connection with the interconnect region;
whereby the bulk conductor of the lead body is electrically connected with the exposed electrode via the interconnect region so as to establish a path for travel of signals between the exposed electrode and the first end of the bulk conductor of the lead body.

2. The method of claim 1, wherein the flexible circuit defines a neural interface and comprises a microfabricated substrate and the exposed electrode.

3. The method of claim 1, wherein the interconnect region comprises metal or other electrically conductive material arranged in an electrically conductive path between a first point and a second point;
wherein the establishing an electrical connection between the interconnect region and the exposed electrode comprises establishing a first electrical connection between the first point of the electrically conductive path of the interconnect region and the exposed electrode; and
wherein the joining the second end of the bulk conductor of the lead body in electrical connection with the interconnect region comprises joining the second end of the bulk conductor of the lead body in a second electrical connection with the second point of the electrically conductive path of the interconnect region.

4. The method of claim 3, further comprising
applying a first insulative coating over the first electrical connection that connects the first point of the electrically conductive path of the interconnect region with the exposed electrode; and
applying a second insulative coating over the second electrical connection that connects the second point of the electrically conductive path of the interconnect region with the lead body.

5. The method of claim 1, wherein the providing the interconnect region comprises providing an interconnect substrate distinct from the flexible circuit, the interconnect substrate comprising the interconnect region; and
wherein the establishing an electrical connection between the interconnect region and the exposed electrode comprises joining the flexible circuit in electrical connection with the interconnect region of the interconnect substrate.

6. The method of claim 1, wherein the providing the interconnect region and the establishing the electrical connection between the interconnect region and the exposed electrode comprises providing the interconnect region as a portion of the flexible circuit and in electrical connection with the exposed electrode.

7. The method of claim 1, wherein the electronic device comprises a component for receiving, transmitting, or receiving and transmitting electrical signals via the bulk conductor.

8. The method of claim 1, wherein the joining the second end of the bulk conductor of the lead body in electrical connection with the interconnect region is accomplished by welding the second end of the bulk conductor to a bond pad of the interconnect region by welding that comprises laser welding, resistance welding, or ultrasonic welding.

9. The method of claim 1, wherein at least one of:
the joining the second end of the bulk conductor of the lead body in electrical connection with the interconnect region is accomplished by wire-bonding the second end of the bulk conductor to a bond pad of the interconnect region; or the establishing an electrical connection between the interconnect region and the exposed electrode is accomplished by wire-bonding the interconnect region with the flexible circuit.

10. The method of claim 1, wherein the interconnect region comprises a multiplexer;
wherein the multiplexer is in electrical connection with the exposed electrode as a result of the establishing the electrical connection between the interconnect region and the exposed electrode;
wherein the multiplexer is in electrical connection with the bulk conductor as a result of the joining the second end of the bulk conductor of the lead body in electrical connection with the interconnect region; and
wherein the multiplexer is configured to control routing or other characteristics of signals relative to the exposed electrode in response to input received via the bulk conductor.

11. A system comprising:
a lead body comprising a bulk conductor having a first end and a second end;
a flexible circuit comprising an exposed electrode; and
an interconnect region disposed between the exposed electrode and the bulk conductor, wherein the exposed electrode is electrically connected to the interconnect region and the bulk conductor is joined in electrical connection with the interconnect region such that the exposed electrode and bulk conductor are in electrical communication via the interconnect region.

12. The system of claim 11, further comprising an electronic device coupled with the lead body for at least one of sending or receiving signals relative to the exposed electrode via the bulk conductor of the lead body.

13. The system of claim 11, wherein the lead body comprises at least one of:
a plurality of lumens each containing a respective bulk conductor; or
a plurality of bulk conductors coiled about a mandrel.

14. The system of claim 11, further comprising an interconnect substrate distinct from the flexible circuit, the interconnect substrate comprising the interconnect region.

15. The system of claim 11, wherein the interconnect region comprises a portion of the flexible circuit.

16. The system of claim 11, further comprising a multiplexer in electrical connection with the exposed electrode and in electrical connection with the bulk conductor, wherein the multiplexer is configured to control characteristics of signals relative to the exposed electrode in response to input received via the bulk conductor.

17. A system comprising:
a lead body comprising a bulk conductor having a first end and a second end;
a flexible circuit comprising an exposed electrode;
an interconnect region disposed between the exposed electrode and the bulk conductor, wherein the exposed electrode is electrically connected to the interconnect region and the bulk conductor is joined in electrical connection with the interconnect region such that the exposed electrode and bulk conductor are in electrical communication via the interconnect region; and
a multiplexer in electrical connection with the exposed electrode and in electrical connection with the bulk conductor, wherein the multiplexer is configured to control characteristics of signals relative to the exposed electrode in response to input received via the bulk conductor.

18. The system of claim 17, wherein the bulk conductor comprises a first bulk conductor configured to provide power to the multiplexer and a second bulk conductor configured to provide control signals to the multiplexer.

19. The system of claim 17, wherein the flexible circuit comprises a plurality of electrodes; and
wherein the multiplexer is configured to control, based on input received through the bulk conductor, at least one of: routing of stimulation signals among the plurality of electrodes;
routing of monitoring signals from among the plurality of electrodes;
a sequential order of transmission of stimulation signals among the plurality of electrodes; or
a sequential order of transmission of monitoring signals from among the plurality of electrodes.

20. The system of claim 17, wherein the multiplexer is configured to control a polarity or other variable characteristic of a stimulation signal to the exposed electrode based on input received through the bulk conductor.
